(19) United States
(12) Patent Application Publication
(10) Pub. No.: US 2025/0100482 A1
(43) Pub. Date: Mar. 27, 2025

(54) VEHICLE OPERATION WITH PHYSIOLOGICAL MONITORING

(71) Applicant: Masimo Corporation, Irvine, CA (US)

(72) Inventors: Ammar Al-Ali, San Juan Capistrano, CA (US); Richard Priddell, Irvine, CA (US); Chad A. DeJong, Los Angeles, CA (US); Stephen Scruggs, Newport Beach, CA (US)

(21) Appl. No.: 18/883,261

(22) Filed: Sep. 12, 2024

Related U.S. Application Data
(60) Provisional application No. 63/626,963, filed on Jan. 30, 2024.
(60) Provisional application No. 63/585,578, filed on Sep. 26, 2023.

(51) Int. Cl. B60R 16/027; A61B 5/00; A61B 5/1455; A61B 5/282; B62D 1/04

(52) U.S. Cl. CPC B60R 16/027; B62D 1/046; A61B 5/14552; A61B 5/282; A61B 5/6893

(57) ABSTRACT

A computing system associated with a vehicle can access sensor data originating from one or more sensors disposed within a vehicle and can access device data originating from a user device. The sensor data and the device data can comprise physiological data of a user of the vehicle. The computing system can determine a physiological status of the user of the vehicle based on at least the sensor data or the device data and cause the vehicle to perform one or more operations based on at least the physiological status of the user.

### CROSS REFERENCE TO RELATED APPLICATIONS

Any and all applications for which a foreign or domestic priority claim is identified in the Application Data Sheet as filed with the present application are hereby incorporated by reference under 37 CFR 1.57 for all purposes and for all that they contain.

### TECHNICAL FIELD

The present disclosure relates to the field of physiological sensors. More specifically, the present disclosure describes, among other things, physiological sensor(s) within a vehicle for monitoring a physiological status of a user of the vehicle.

### BACKGROUND

Sensors, devices, and/or monitors can collect or analyze a user's (which may also be referred to as a “subject”, “wearer,” “individual” or “patient”) physiological parameters such as blood oxygen saturation level, temperature, respiratory rate, pulse rate, blood pressure, and the like. Such devices can include, for example, acoustic sensors, electroencephalogram (EEG) sensors, electrocardiogram (ECG) devices, blood pressure monitors, temperature sensors, and pulse oximeters, among others. Various sensors/devices (such as those just mentioned) can be attached to a patient and connected to one or more patient monitoring devices using cables or via wireless connection. Vehicles can include steering wheels. A user can operate a vehicle such as by handling the steering wheel of the vehicle. A vehicle operator may sit within the vehicle and place their hands on the steering wheel to control the vehicle.

### SUMMARY

The present disclosure provides one or more sensor modules disposed within a vehicle, such as on a door, a center console, or a steering wheel of the vehicle. The sensor modules can include a temperature sensor, a pulse oximeter, and/or ECG electrodes. The sensor module(s) may monitor physiological data of the user, such as pulse rate, heart rate, blood oxygen saturation, cardiac arrythmias, or the like. The vehicle may automatically adjust one or more functions of the vehicle (e.g., for the user's comfort or safety) based on the user's physiological data obtained by the sensor module(s). Advantageously, a user's physiological conditions may be monitored as the user drives.

Disclosed herein is a steering apparatus configured for physiological monitoring that can comprise and a left portion and a right portion that are configured to rotate about a central portion of the steering apparatus to control the vehicle. The left portion can be symmetrical with the right portion across the central portion. The left portion can comprise a depressed region forming a curved surface that is shaped to receive a digit of a left hand of a user when the user places their left hand on the steering apparatus. The curved surface can be elongate, extending along a length of the left portion, wherein a first sensor is positioned within the curved surface. The right portion can comprise another depressed region forming another curved surface on the right portion that is shaped to receive a digit of a right hand of the user when the user places their right hand on the steering apparatus. The other curved surface can extend along a length of the right portion, wherein a second sensor is positioned within the another curved surface.

In some implementations, a controller is configured to control an operation on the vehicle based on the physiological characteristics of the user determined from sensor data from the first sensor or the second sensor.

In some implementations, the curved surface comprises a left arm (LA) electrode configured to contact the digit of the left hand and respond to electrical voltages conducted through the left hand of the user to the LA electrode, wherein the other curved surface comprises a right arm (RA) electrode configured to contact the digit of the right hand of the user and respond to electrical voltages conducted through the right hand to the RA electrode, the RA electrode being operably coupled with the LA electrode to form an electrode pair, the RA electrode and the LA electrode being physically and electrically isolated from each other on the steering apparatus.

In some implementations, the steering apparatus comprises an oximeter positioned with the depressed region of the left portion, the oximeter comprising one or more optical emitters configured to emit optical radiation away from the curved surface toward the digit of the left hand of the user, the oximeter comprising one or more optical detectors configured to generate plethysmography data responsive to detecting optical radiation attenuated by the tissue of the user.

In some implementations, the steering apparatus comprises an emitter chamber embedded within the curved surface housing the one or more optical emitters; a detector chamber embedded within the curved surface housing the one or more optical detectors; and a light barrier positioned on the curved surface between the emitter chamber and the detector chamber. The light barrier can extend from the curved surface and can be configured to: induce optical radiation emitted from the one or more optical emitters to penetrate the digit of the user before arriving at the optical detector; and inhibit optical radiation emitted from the one or more optical emitters from travelling within a gap between the curved surface and the digit of the left hand of the user.

In some implementations, the LA electrode at least partially surrounds the one or more emitters and/or the one or more detectors.

In some implementations, the steering apparatus comprises a second left arm (LA) electrode positioned on the curved surface of the depressed region, the second LA electrode configured to contact the digit of the left hand and respond to the electrical voltage conducted through the left hand of the user to the second LA electrode.

In some implementations, the curved surface on the left portion and the other curved surface on the right portion are symmetrical with each other across the central portion.

In some implementations, the steering apparatus comprises a temperature sensor positioned with the depressed region of the left portion.

In some implementations, a length of the curved surface is between 20 mm and 60 mm.

In some implementations, a width of the curved surface is between 15 mm and 35 mm.

The steering apparatus of claim 1 wherein the curved surface is bounded by a stadium shaped perimeter, wherein a width of the curved surface is between 60% and 65% of a length of the curved surface.

In some implementations, the depressed region is positioned on a top portion of the steering apparatus.

In some implementations, the depressed region is positioned on a front of the steering apparatus facing toward the user.

In some implementations, the depressed region is positioned on a back of the steering apparatus facing away from the user.

Disclosed herein is a physiological sensor that can comprise a curved surface shaped to receive a digit of a user; an emitter chamber housing an optical emitter, the emitter chamber having an emitter window disposed on a portion of the curved surface for optical radiation to pass through; a detector chamber housing an optical detector, the detector chamber having a detector window disposed on another portion of the curved surface for optical radiation to pass through; and a light barrier positioned on the curved surface between the emitter chamber and the detector chamber. The light barrier can extend from the curved surface and can be configured to: induce optical radiation emitted from the optical emitter to penetrate the digit of the user before arriving at the optical detector; and inhibit optical radiation emitted from the optical emitter from travelling between the digit and the curved surface between the emitter chamber and the detector chamber.

In some implementations, the detector window is oriented toward the emitter window, the optical emitter and the optical detector configured to perform reflectance-based oximetry.

In some implementations, the emitter window and the detector window form non-planar portions of the curved surface.

In some implementations, the physiological sensor can comprise a first electrode having a portion thereof flush with the curved surface and exposed for contact with the user, the first electrode at least partially surrounding the detector chamber; and a second electrode having a portion thereof flush with the curved surface and exposed for contact with the user, the second electrode at least partially surrounding the emitter chamber.

In some implementations, the first electrode is symmetrical to the second electrode across the light barrier.

In some implementations, the physiological sensor can comprise a temperature sensor chamber housing a temperature sensor, the temperature sensor chamber embedded within the curved surface.

In some implementations, the curved surface is symmetrical about a midline, wherein the midline intersects the emitter chamber.

In some implementations, the curved surface is symmetrical about a midline, wherein the midline intersects the detector chamber.

In some implementations, the physiological sensor is positioned on a portion of a vehicle.

Disclosed herein is a physiological monitoring system within a vehicle to enhance a user's driving experience. The system can comprise: a first sensor module having a left arm (LA) electrode positioned on a left side of a steering wheel of a vehicle and configured to contact a digit on a left hand of a user when the user places their left hand on the steering wheel, the first sensor module configured to generate left voltage data responsive to electrical voltage conducted through the left hand of the user to the LA electrode; a second sensor module having a right arm (RA) electrode positioned on a right side of the steering wheel and configured to contact a digit on a right hand of the user when the user places their right hand on the steering wheel, the second sensor module configured to generate right voltage data responsive to electrical voltage conducted through the right hand of the user to the RA electrode; and one or more hardware computer processors configured to: determine cardiac characteristics of the user from the left voltage data and the right voltage data; and cause the vehicle to perform one or more operations based on the cardiac characteristics of the user to enhance a vehicle operating experience of the user.

In some implementations, the first sensor module and the second sensor module are positioned on the steering wheel at mirror images of each other.

In some implementations, a length of the first sensor module extends along a perimeter of the steering wheel in a direction that is tangential to the perimeter of the steering wheel.

In some implementations, a length of the first sensor module extends along a perimeter of the steering wheel in a direction that is perpendicular to the perimeter of the steering wheel.

A computing system can comprise one or more hardware computer processors which can be configured to execute program instructions to cause the computing system to: access sensor data originating from one or more sensors disposed within a vehicle, the sensor data comprising physiological data of a user of the vehicle; access device data originating from a user device, the device data comprising physiological data of the user of the vehicle, at least a portion of the device data originating from one or more physiological sensors of the user device; determine a physiological status of the user of the vehicle based on at least the sensor data or the device data; and cause the vehicle to perform one or more operations based on at least the physiological status of the user.

In some implementations, the sensor data comprises data relating to an operation of the vehicle including one or more of vehicle linear acceleration, vehicle angular acceleration, vehicle velocity, proximity of the vehicle to other vehicles, proximity of the vehicle to road lines, distance traveled, and a destination.

In some implementations, the one or more hardware computer processors are embodied in the vehicle.

In some implementations, the one or more hardware computer processors are embodied in a server remote to the vehicle.

In some implementations, the one or more hardware computer processors are embodied in the user device.

In some implementations, the one or more operations include adjusting one or more cabin settings including a cabin temperature, an audio setting, a cabin scent, a seat position, a seat operation, or a pedal position.

In some implementations, the one or more operations include preventing the vehicle from starting.

In some implementations, the one or more operations include activating one or more autonomous driving features.

In some implementations, the one or more operations include initiating a phone call.

In some implementations, the one or more operations include generating directions to a healthcare facility.

In some implementations, the one or more operations include generating one or more alerts.

In some implementations, the one or more operations include generating user interface data for displaying one or more physiological parameters on a display disposed within the vehicle and/or on the user device.

In some implementations, the one or more operations include deactivating an airbag to prevent the airbag from deploying.

In some implementations, the one or more hardware computer processors are further configured to execute the program instructions to cause the computing system to: determine an identity of the user of the vehicle based on at least the sensor data or the device data; and perform the one or more operations based on at least the identity of the user of the vehicle.

In some implementations, the physiological status of the user of the vehicle includes one or more of pulse rate, heart rate, respiration rate, blood oxygen saturation, blood glucose level, perspiration level, and body temperature.

In some implementations, the physiological status of the user of the vehicle includes one or more of user head position, distance between a steering wheel and a head of the user, eye movement, eye status, pupil dilation, and hand gestures.

In some implementations, the physiological status of the user of the vehicle includes an alertness level of the user of the vehicle, a drowsiness level of the user of the vehicle, and an intoxication level of the user of the vehicle.

In some implementations, the one or more sensors disposed within the vehicle include one or more of a physiological sensor, a camera, a proximity sensor, a radar sensor, a microwave sensor, an infrared sensor, a light sensor, and a temperature sensor.

In some implementations, the user device includes one or more of a phone, a wearable device, a watch, an auricular device, a physiological sensor, and an insulin pump.

In some implementations, the one or more hardware computer processors are further configured to execute the program instructions to cause the computing system to: communicate the sensor data to a remote computing device comprising one or more of a server or the user device.

A physiological sensor can comprise: a surface being bisected by a midline, the surface being symmetrical about the midline, the surface being concave; an optical emitter positioned on the midline of the surface, the optical emitter configured to emit optical radiation having one or more wavelengths; an optical detector positioned on the surface, the optical detector configured to generate detector signals responsive to detecting a portion of the optical radiation from the optical emitter having travelled through tissue of a user; and a light barrier positioned on the surface between the optical emitter and the optical detector, the light barrier extending along the surface perpendicular to the midline, the light barrier comprising an opaque material configured to inhibit transmission of another portion of the optical radiation emitted from the optical emitter.

In some implementations, the physiological sensor is positioned on a portion of a vehicle.

In some implementations, the physiological sensor can further comprise a temperature sensor positioned on the midline of the surface, the light barrier being positioned between the temperature sensor and the optical emitter.

In some implementations, the physiological sensor can further comprise: a first electrode positioned on the surface adjacent to the optical emitter, the first electrode being symmetrical about the midline, the first electrode being configured to conduct electrical voltages originating from a user; and a second electrode positioned on the surface adjacent to the optical detector, the second electrode being symmetrical about the midline, the second electrode being configured to conduct electrical voltages originating from the user.

In some implementations, the first electrode is symmetrical to the second electrode about the light barrier.

In some implementations, the first electrode is positioned on a same side of the light barrier as the optical emitter, wherein the second electrode is positioned on a same side of the light barrier as the optical detector.

A physiological sensor can comprise: a surface being bisected by a midline, the surface being symmetrical about the midline, the surface being concave; and an electrode positioned on the surface, the electrode being symmetrical about the midline, the electrode being positioned on a first side of the surface, the electrode comprising a circular perimeter, the electrode being configured to conduct electrical voltages originating from the user.

In some implementations, the physiological sensor does not comprise a temperature sensor or a pulse oximeter or another electrode.

In some implementations, the physiological sensor does not comprise another electrode.

In some implementations, the physiological sensor is positioned on a portion of a vehicle.

A physiological monitoring system for monitoring a user of a vehicle can comprise: a first sensor module, a second sensor module, and a hardware processor in communication with the first sensor module and the second sensor module. The first sensor module can be positioned at a first location on a steering wheel of a vehicle. The first sensor module can comprise a temperature sensor, an emitter, a detector, and one or more electrodes. The second sensor module can be positioned at a second location on the steering wheel of the vehicle. The second sensor module can comprise one or more electrodes. The hardware processor can be in communication with the first sensor module and the second sensor module and can be configured to: access first physiological data originating from the first sensor module; access second physiological data originating from the second sensor module; determine one or more physiological conditions of a user of the vehicle based on at least the first physiological data and/or the second physiological data; and perform one or more operations based on at least the physiological condition of the user.

In some implementations, the one or more operations comprise one or more of adjusting a lighting of the vehicle, adjusting a temperature of the vehicle, adjusting a driving operation of the vehicle, adjusting an audio of the vehicle, verifying a user identity, verifying a user's permission to operate the vehicle, displaying indicia of the physiological condition to the user, or generating an alert.

Various combinations of the above and below recited features, embodiments, implementations, and aspects are also disclosed and contemplated by the present disclosure.

Additional implementations of the disclosure are described below in reference to the appended claims, which may serve as an additional summary of the disclosure.

In various implementations, systems and/or computer systems are disclosed that comprise a computer-readable storage medium having program instructions embodied therewith, and one or more processors configured to execute the program instructions to cause the systems and/or computer systems to perform operations comprising one or more aspects of the above-and/or below-described implementations (including one or more aspects of the appended claims).

In various implementations, computer-implemented methods are disclosed in which, by one or more processors executing program instructions, one or more aspects of the above-and/or below-described implementations (including one or more aspects of the appended claims) are implemented and/or performed.

In various implementations, computer program products comprising a computer-readable storage medium are disclosed, wherein the computer-readable storage medium has program instructions embodied therewith, the program instructions executable by one or more processors to cause the one or more processors to perform operations comprising one or more aspects of the above-and/or below-described implementations (including one or more aspects of the appended claims).

### BRIEF DESCRIPTION OF THE DRAWINGS

Various implementations will be described hereinafter with reference to the accompanying drawings. These implementations are illustrated and described by example only, and are not intended to limit the scope of the disclosure. In the drawings, similar elements may have similar reference numerals.

FIGS. 1A-1B illustrate an example physiological sensor module.

FIGS. 2A-2B illustrate another example physiological sensor module.

FIGS. 3-4 are perspective views of sensor modules.

FIGS. 5A-5B illustrate a user touching a sensor module.

FIG. 6A is a cross-section view of the sensor module taken along the line illustrated in FIG. 1A.

FIG. 6B is a cross-section view of the sensor module taken along the line illustrated in FIG. 1A shown with a finger.

FIG. 6C is a cross-section of the sensor module taken along the line illustrated in FIG. 1A.

FIG. 6D is a cross-section view of the other sensor module taken along the line illustrated in FIG. 2A.

FIGS. 7A-7B are front views of a steering wheel with example implementations of sensor modules.

FIGS. 7C-7D are back views of a steering wheel with example implementations of sensor modules.

FIG. 8 is a block diagram illustrating an example sensor module.

FIG. 9 is a schematic diagram of various computing devices in communication over a network including an example implementation of a vehicle system having sensor(s).

FIG. 10 illustrates an example implementation of sensors in a vehicle.

### DETAILED DESCRIPTION

The present disclosure will now be described with reference to the accompanying figures, wherein like numerals may refer to like elements throughout. The following description is merely illustrative in nature and is in no way intended to limit the disclosure, its application, or uses. It should be understood that steps within a method may be executed in different order without altering the principles of the present disclosure. Furthermore, the devices, systems, and/or methods disclosed herein can include several novel features, no single one of which is solely responsible for its desirable attributes or which is essential to practicing the devices, systems, and/or methods disclosed herein. Additionally, the structures, systems, and/or devices described herein may be embodied as integrated components or as separate components.

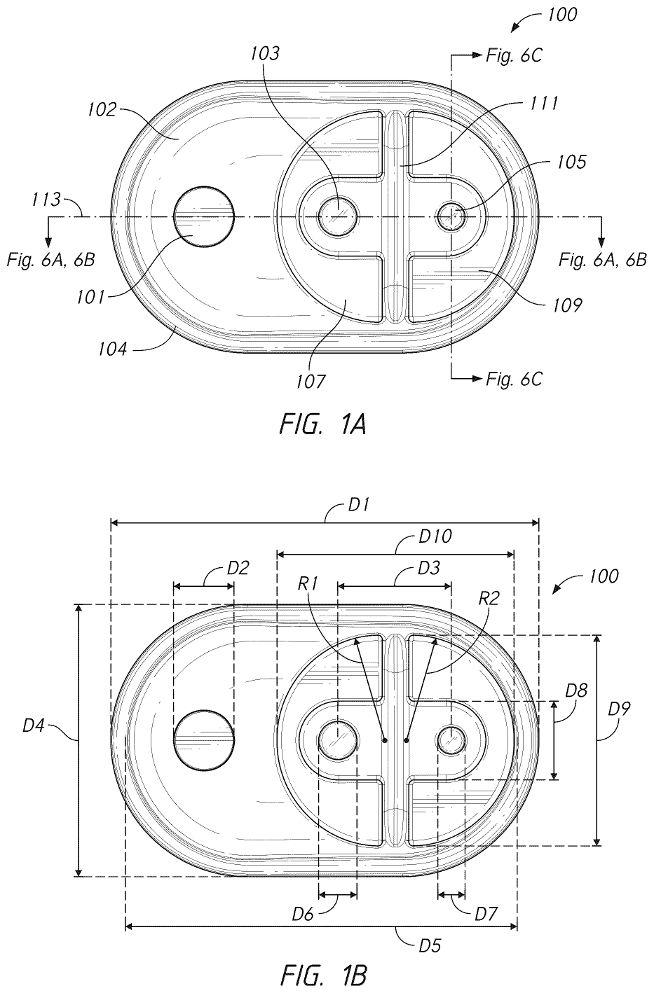

FIG. 1A is a front view of an example physiological sensor module 100, which may also be referred to as a physiological sensor or a sensor housing. The sensor module 100 can include a temperature sensor 101, an detector chamber 103, an emitter chamber 105, a first electrode 107, a second electrode 109, and a light barrier 111. In some implementations, sensor module 100 includes only a single electrode. In some implementations, sensor module 100 may not include any electrodes. In some implementations, sensor module 100 may not include a temperature sensor. In some implementations, sensor module 100 may not include an emitter chamber and/or detector chamber (or emitters/detectors). In some implementations, the sensor module 100 includes one or more hardware processors. In some implementations, the sensor module 100 can communicate with one or more hardware processors remote to the sensor module 100 such as processors disposed within a vehicle and/or within a user device such as watch or phone.

The emitter chamber 105 can house one or more emitters configured to emit optical radiation. The detector chamber 103 can house one or more detectors configured to detect optical radiation. The light barrier 111 can be positioned between the emitter chamber 105 and the detector chamber 103. The temperature sensor 101 may be positioned on a same side of the light barrier 111 as the detector chamber 103. The temperature sensor 101 may be positioned on an opposite side of the light barrier 111 as the emitter chamber 105. The detector chamber 103 may be positioned between the temperature sensor 101 and the emitter chamber 105. The barrier 111 may be positioned between the temperature sensor 101 and the emitter chamber 105. Advantageously, separating the temperature sensor 101 from the emitter chamber 105 (such as by placing the detector chamber 103 between the temperature sensor 101 and the emitter chamber 105) may improve an accuracy of the temperature sensor 101 measurements such as by reducing an effect that heat generated by the emitter chamber 105 (e.g., heat generating from driving LEDs) may have on the temperature sensor 101.

The light barrier 111 can be positioned between the first electrode 107 and the second electrode 109. The first electrode 107 may be symmetrical to the second electrode 109 about the light barrier 111. The first electrode 107 may be positioned adjacent to the detector chamber 103 (e.g., on a same side of the light barrier 111 as the detector chamber 103). The first electrode 107 may at least partially surround the detector chamber 103 and/or enclose the detector 103 adjacent to the barrier 111. The first electrode 107 may form a substantially semi-annulus. The second electrode 109 may be positioned adjacent to the emitter chamber 105 (e.g., on a same side of the light barrier 111 as the emitter chamber 105). The second electrode 109 may at least partially surround the emitter chamber 105 and/or enclose the emitter chamber 105 adjacent to the barrier 111. The second electrode 109 may form a substantially semi-annulus.

The electrodes 107, 109 can be formed of an electrically conductive material such as metal or metal alloy. The electrodes 107, 109 can contact the skin of a user and can conduct electrical signals conducted through the tissue of the user. The electrical signals may have originated at the user's heart and may be associated with cardiac activity of the user. The first electrode 107 may operate as (or be referred to as) a right leg drive (RLD) electrode, a ground electrode, or a reference electrode. The second electrode 109 may operate as (or be referred to as) a right arm (RA) electrode (or left arm (LA) electrode), or positive electrode (or negative electrode). The first electrode 107 may contact a same digit of the user as the second electrode 109 at the same time.

The temperature sensor 101 may contact the skin of a user. The temperature sensor 101 may be in proximity to the skin of a user. The temperature sensor 101 may comprise one or more of a thermistor, infrared sensor, thermocouple, resistive sensor, or the like. The temperature sensor 101 can generate temperature data indicating a temperature of a user.

The sensor module 100 may comprise a surface 102. The temperature sensor 101, detector chamber 103, emitter chamber 105, first electrode 107, second electrode 109, and/or light barrier 111 may be positioned, at least partially, on the surface 102. At least a portion of the surface 102 may be curved, such as concave. For example, the surface 102 may be recessed from a perimeter 104 of the sensor module 100. The surface 102 may be sized and/or shaped to receive a portion of a body of a user, such as a finger or thumb. The surface 102 may be sized and/or shaped to facilitate initiating contact between the skin of a user and one or more of the temperature sensor 101, the first electrode 107, second electrode 109, the detector chamber 103, and the emitter chamber 105 which can improve accuracy of physiological measurements. The perimeter 104 can be a stadium shape that can bound the curved surface 102.

Sensor module 100 is shown with a midline 113 superimposed thereon. The midline 113 may bisect the sensor module 100. The sensor module 100 may be symmetrical about the midline 113. The temperature sensor 101 may be positioned on the midline 113. The midline 113 may bisect the temperature sensor 101 such that the temperature sensor 101 is symmetrical about the midline 113. The detector chamber 103 may be positioned on the midline 113. The midline 113 may bisect the detector chamber 103 such that the detector chamber 103 is symmetrical about the midline 113. The emitter chamber 105 may be positioned on the midline 113. The midline 113 may bisect the emitter chamber 105 such that the emitter chamber 105 is symmetrical about the midline 113. The electrode 107 may be positioned on the midline 113. The midline 113 may bisect the electrode 107 such that the electrode 107 is symmetrical about the midline 113. The electrode 109 may be positioned on the midline 113. The midline 113 may bisect the electrode 109 such that the electrode 109 is symmetrical about the midline 113. The barrier 111 may extend along the surface 102 perpendicular to the midline 113. The midline 113 may bisect the barrier 111 such that the barrier 111 is symmetrical about the midline 113.

FIG. 1B is a front view of the sensor module 100 illustrating dimensions of various aspects of the sensor module 100. The length of sensor module 100 is represented by D1. D1 may be between 20 mm and 60 mm, between 30 mm and 50 mm, between 35 mm and 45 mm, or between 38 mm and 42 mm. In some implementations, D1 may be about 39 mm. The width or diameter of the temperature sensor 101 is represented by D2. D2 may be between about 3 mm and 7 mm, between 4 mm and 6 mm, between 5 mm and 6 mm, or about 5.5 mm. The distance between the detector chamber 103 and the emitter chamber 105 (e.g., a distance between the centers of the detector chamber 103 and emitter chamber 105) is represented by D3. D3 may be between 7 mm and 13 mm, between 8 mm and 12 mm, between 9 mm and 11 mm, or about 10 mm. The width of the sensor module 100 is represented by D4. D4 may be less than D1. D4 may be between 40% and 90% of D1, between 50% and 80% of D1, between 60% and 70% of D1, or between 60% and 65% of D1. D4 may be between 15 mm and 35 mm, between 20 mm and 30 mm, or about 25 mm. The length of the sensor module 100 inside the perimeter 104 (e.g., the length of the surface 102) is represented by D5. D5 may be less than D1. D5 may be between 20 mm and 50 mm, between 25 mm and 45 mm, between 30 mm and 40 mm, or about 35 mm. A width (e.g., diameter) of detector chamber 103 is represented by D6. D6 may be between 2 mm and 5 mm, between 2.5 mm and 4.5 mm, between 3 mm and 4 mm, or about 3.5 mm, or about 3.75 mm. A width (e.g., diameter) of emitter chamber 105 is represented by D7. D7 may be less than D6. D7 may be between 1 mm and 4 mm, between 1.5 mm and 3.5 mm, between 2 mm and 3 mm, or about 2.5 mm. D8 may refer to a width of first electrode 107 and/or second electrode 109. D8 may be between 5 mm and 9 mm, between 6 mm and 8 mm, between 6.5 mm and 7.5 mm, or about 7.2 mm. A width (e.g., diameter) of first electrode 107 and/or second electrode 109 is represented by D9. D9 may be between 16 mm and 21 mm, between 17 mm and 20 mm, between 18 mm and 20 mm, or about 19.35 mm. A radius of a circle partially defined by the first electrode 107 is represented by R1. R1 extends away from the center of the circle partially defined by the first electrode 107 and the center is located adjacent to the barrier 111. R1 may be half of D9. R1 may be between 7 mm and 12 mm, between 8 mm and 11 mm, between 9 mm and 10 mm, or about 9.7 mm. A radius of a circle partially defined by the second electrode 109 is represented by R2. R2 extends away from the center of the circle partially defined by the second electrode 109 and the center is located adjacent to the barrier 111. R2 may be half of D9. R2 may be the same length as R1. R2 may be between 7 mm and 12 mm, between 8 mm and 11 mm, between 9 mm and 10 mm, or about 9.7 mm. The length of the first electrode 107 in combination with the second electrode 109 is represented by D10. D10 may be greater than D9. D10 may be between 18 mm and 25 mm, between 19 mm and 24 mm, between 20 mm and 23 mm, between 21 mm and 22 mm, or about 21.7 mm.

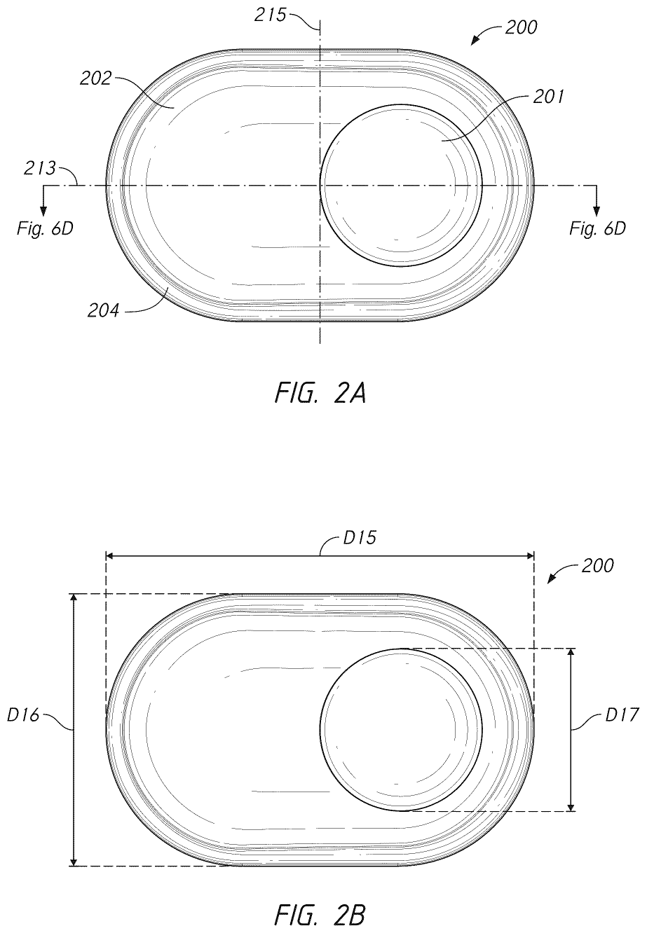

FIG. 2A is a front view of another example physiological sensor module 200, which may also be referred to as a physiological sensor or a sensor housing. The sensor module 200 can include an electrode 201. The electrode 201 may operate as (or be referred to as) a left arm (LA) electrode (or right arm (RA) electrode), or positive electrode (or negative electrode). The electrode 201 may contact a different portion of the user's body (e.g., different finger/thumb) than the first electrode 107 or the second electrode 109. For example, the electrode 201 can contact a finger on one hand of a user while the first electrode 107 and/or second electrode 109 contact a finger on another hand of the user. The electrode 201 can conduct electrical signals conducted via the skin of a user, which may have originated from cardiac activity of the user. The electrode 201 may be circular. For example, a perimeter of the electrode 201 may define a circle. The electrode 201 may be non-planar. For example, a surface of the electrode 201 can be curved such that a center portion of the electrode 201 is recessed from an outer perimeter of the electrode 201. The electrode 201 may be sized and/or shaped to receive a digit of a user. In some implementations, sensor module 200 may comprise a plurality of electrodes.

The sensor module 200 may comprise a surface 202. The electrode 201 may be positioned, at least partially, on the surface 202. At least a portion of the surface 102 may be curved, such as concave. For example, the surface 202 may be recessed from a perimeter 204 of the sensor module 200. The surface 202 may be sized and/or shaped to receive a portion of a body of a user such as a finger or thumb. The surface 202 may be sized and/or shaped to facilitate initiating contact between the skin of a user and the electrode 201 which can improve physiological measurements of the user.

Sensor module 200 is shown with midline 213 and midline 215 superimposed thereon. For ease of reference, midline 213 may be referred to as horizontal midline 213 because it is illustrated as being horizontal with respect to the page. For ease of reference, midline 215 may be referred to as vertical midline 215 because it is illustrated as being vertical with respect to the page. The horizontal midline 213 may be perpendicular to the vertical midline 215. The horizontal midline 213 may bisect the sensor module 200. The sensor module 200 may be symmetrical about the horizontal midline 213. The electrode 201 may be positioned on the horizontal midline 213. The horizontal midline 213 may bisect the electrode 201 such that the electrode 201 is symmetrical about the horizontal midline 213. The vertical midline 215 may bisect the sensor module 200. The sensor module 200 may be symmetrical about the vertical midline 215 except for the presence of the electrode 201. The electrode 201 may not be positioned on the vertical midline 215 such that the vertical midline 215 may not intersect the electrode 201. The vertical midline 215 may be tangential to a perimeter of the electrode 201.

In some implementations, the sensor module 200 may be a same size and/or shape as sensor module 100 shown and/or described herein. In some implementations, the sensor module 200 may comprise one or more of a temperature sensor, emitter, and/or detector.

FIG. 2B is a front view of the sensor module 200 illustrating dimensions of various aspects of the sensor module 200. A length of sensor module 200 is represented by D15. D15 may be the same length as D1 such that the sensor module 200 is a same length as the sensor module 100. A width of the sensor module 200 is represented by D16. D16 may be the same length as D4 such that a width of the sensor module 200 is the same as the width of sensor module 100. A width (e.g., diameter) of electrode 201 is represented by D17. D17 may be between 12 mm and 17, between 13 mm and 16 mm, between 14 mm and 15 mm, or about 14.9 mm. D17 may be less than D9 and/or D10.

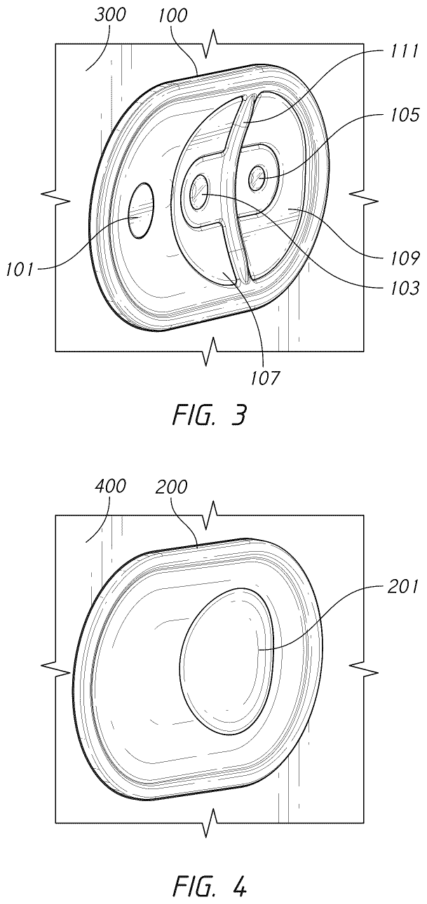

FIG. 3 is a perspective view of the sensor module 100 illustrating the temperature sensor 101, detector chamber 103, emitter chamber 105, first electrode 107, second electrode 109, and light barrier 111. The sensor module 100 is shown positioned on (or at least partially embedded within) a structure 300 which may be a portion of vehicle such as a door, steering wheel, center console, dashboard, etc.

FIG. 4 is a perspective view of the sensor module 200 illustrating the electrode 201. The sensor module 200 is shown positioned on (or at least partially embedded within) a structure 400 which may be a portion of vehicle such as a door, steering wheel, center console, dashboard, etc.

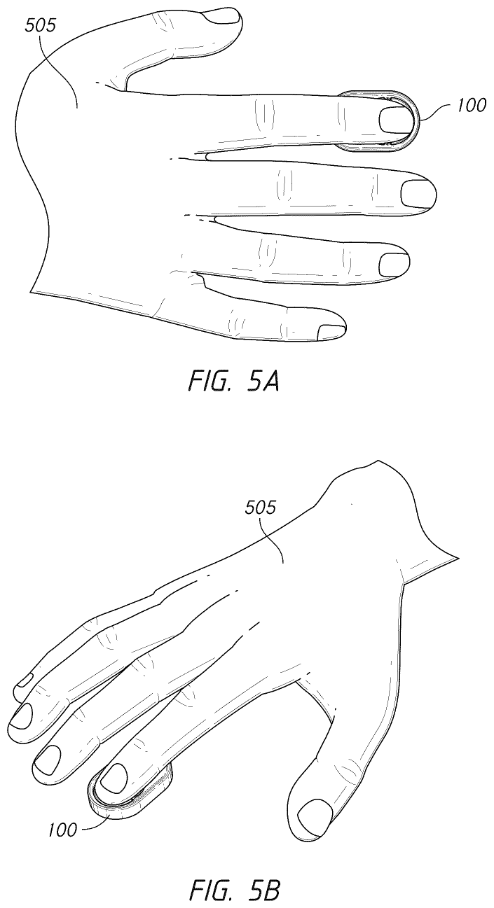

FIGS. 5A-5B illustrate an example implementation of the sensor module 100. A user 505 may position any one of their fingers/thumbs on the sensor module 100. One or more components of the sensor module 100, such as the temperature sensor 101, the detector chamber 103, the emitter chamber 105, the first electrode 107, and/or the second electrode 109 may contact the finger of the user 505. The user 505 may similarly place their finger on the sensor module 200.

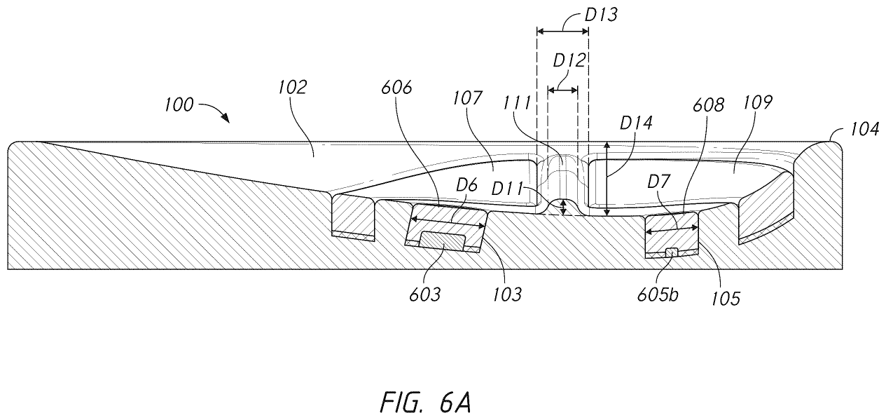

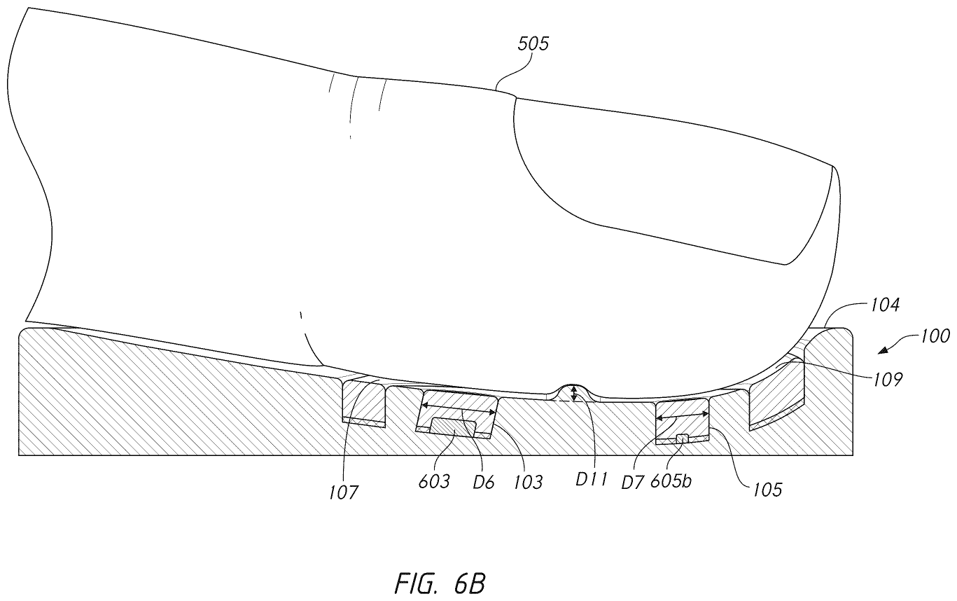

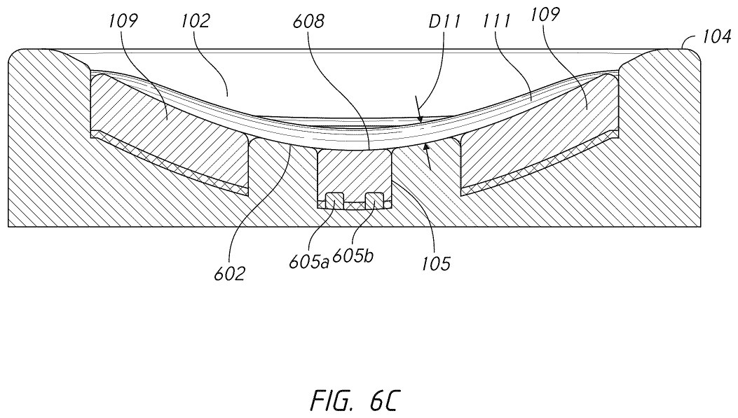

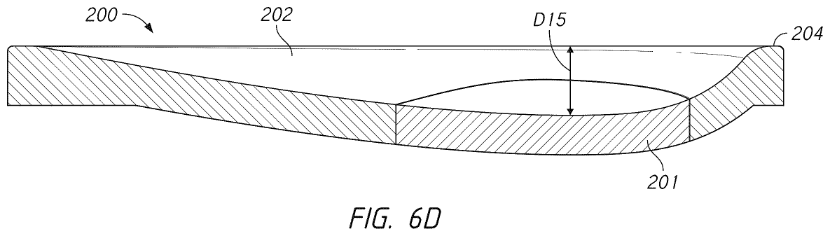

FIG. 6A is a cross-section view of the sensor module 100 taken along the line illustrated in FIG. 1A. The emitter chamber 105 can house one or more optical emitters 605 (e.g., 605a, 605b). The emitters 605 can be positioned within the emitter chamber 105 beneath the surface 102. The emitters 605 can emit electromagnetic radiation having one or more wavelengths such as visible light, infrared, and/or near infrared radiation. The emitters 605 can include one or more light emitting diodes (LEDs). The emitter chamber 105 can include an emitter window 608 configured to allow optical radiation to pass therethrough. The emitter window 608 can include a radiation diffusing material. The emitter window 608 can be formed of a transparent and/or translucent material, such as glass, plastic, and/or polycarbonate. At least a portion of the emitter window 608 can be flush with the surface 102 and can form a portion of the surface 102. A surface area of the emitter window 608 can be a same area as a cross sectional area of the emitter chamber 105. For example, a diameter of the emitter window 608 can be the same size as the diameter of the emitter chamber 105 (represented by D7). In some implementations, the diameter of the emitter window 608 may be less than the diameter D7 of the emitter chamber 105. A perimeter of the emitter window 608 can be beveled. In some implementations, the emitter chamber 105 can house a temperature sensor.

The detector chamber 103 can house one or more detectors 603. The detector 603 can be positioned within the detector chamber 103 beneath the surface 102. The detector 603 can detect electromagnetic radiation such as visible light, infrared, near-infrared, etc. The detector 603 may detect optical radiation emitted by the emitter 605 after it has passed through the tissue of a user and has been attenuated, scattered, and/or absorbed by the tissue of the user. The detector 603 may comprise one or more photodiodes. The detector 603 can generate one or more detector signals responsive to detecting optical radiation. The detector chamber 103 can include a detector window 606 configured to allow optical radiation to pass therethrough. The detector window 606 can include a radiation diffusing material. The detector window 606 can be formed of a transparent and/or translucent material, such as glass, plastic, and/or polycarbonate. At least a portion of the detector window 606 can be flush with the surface 102 and can form a portion of the surface 102. A surface area of the detector window 606 can be a same area as a cross sectional area of the detector chamber 103. For example, a diameter of the detector window 606 can be the same size as the diameter of the detector chamber 103 (represented by D6). In some implementations, the diameter of the detector window 606 may be less than the diameter D6 of the detector chamber 103. A perimeter of the detector window 606 can be beveled. In some implementations, the detector chamber 103 can house a temperature sensor.

The emitter window 608 can be non-planar with the detector window 606. For example, the emitter window 608 and detector window 606 may form a portion of the surface 102 and because the surface 102 is curved, the emitter window 608 and the detector window 606 may not lie in the same plane as each other. The walls of the detector chamber 103 can be non-parallel with the walls of the emitter chamber 105. The detector chamber 103 can be oriented toward the emitter chamber 105.

The surface 102 may recess away from the perimeter 104. The surface 102 may be curved and/or concave such that the surface 102 slopes away from the perimeter 104. Thus, a distance extending vertically (with respect to the page) from the surface 102 to a height defined by the perimeter 104 can vary. For example, a maximum distance from the surface 102 to a height of the perimeter is represented by D14 which can be between 1.5 mm and 3.5 mm, between 2 mm and 3 mm, or about 2.5 mm. D14 can extend to a point on the surface 102 between the light barrier 111 and the emitter chamber 105. Thus, the point of the surface 102 furthest from the level defined by the perimeter 104 may be between the light barrier and the emitter chamber 105.

At least a portion of the first electrode 107 and/or second electrode 109 can be flush with the surface 102 and can form a portion of the surface 102. The light barrier 111 can be positioned on the surface 102 between the detector chamber 103 and the emitter chamber 105. The light barrier 111 can be positioned on the surface 102 between the first electrode 107 and the second electrode 109. The first electrode 107 can be separated from the second electrode 109 by a distance D13. D13 can be between 1.0 mm and 4.0 mm, between 1.5 mm and 3.5 mm, between 2.0 mm and 3.0 mm, or about 2.4 mm. The width of the light barrier 111 may be equal to and/or less than D13.

The width of the light barrier 111 can be non-uniform. For example, the width of the light barrier 111 can decrease with distance from the surface 102 such that a width of the top of the light barrier 111 is less than a width of the bottom of the light barrier 111. The width of the light barrier 111 may vary at least because the light barrier 111 can be beveled. The width of the light barrier 111 is represented by D12 which can be between 0.5 mm and 2.5 mm, between 1.0 mm and 2.0 mm, between 1.25 mm and 1.75 mm, or about 1.4 mm.

The light barrier 111 can protrude from the surface 102 a distance D11. D11 can be between 0.25 mm and 1.25 mm, between 0.5 mm and 1.0 mm, between 0.6 mm and 0.8 mm, or about 0.68 mm.

FIG. 6B is a cross section view of the sensor module 100 taken along the line illustrated in FIG. 1A shown with the finger of a user 505 placed on the sensor module 100. The sensor module 100 may be sized and/or shaped such that the finger of the user 505 may contact the detector chamber 103, detector window 606, emitter chamber 105, emitter window 608, first electrode 107, and/or second electrode 109 when their finger is placed on the sensor module 100. For example, the surface 102 can be curved such that it corresponds to a shape of a human finger. Advantageously, the shape of the surface 102 can facilitate physical contact between the user 505 and the sensors which can improve physiological measurements.

The detector 603 and the emitter(s) 605 can operate as a pulse oximeter, such as a reflectance-based pulse oximeter. For example, optical radiation emitted from the emitters 605 can penetrate the user 505, be attenuated and reflected by the user 505, and can then be detected by the detector 603. In some implementations, the detector 103 and the emitter 105 may operate as a transmittance-based pulse oximeter which may receive a portion of a body part (e.g., a finger, ear, etc.) and enclose the body such that the detector and emitter are on opposite sides of the body part.

The finger of the user 505 may not uniformly contact the surface 102 such that gaps may exist between the finger of the user 505 and the surface 102 (or other portions of the sensor module 100). Light emitted from the emitter 605 can travel through these gaps (e.g., between the finger and the surface 102) without penetrating the finger of the user 505 and can eventually reach the detector 603. This phenomenon may be referred to as light piping and can reduce the accuracy of physiological measurements at least because the detector would be detecting radiation from the emitter 605 that was not attenuated by the tissue of the of the user 505. The light barrier 111 can reduce and/or eliminate light piping. For example, the light barrier 111 can inhibit optical radiation (e.g., visible light, infrared, near-infrared) emitted from the emitters 605 from reaching the detector 603 without penetrating the tissue of the user 505 and being attenuated by the tissue of the user 505. Moreover, the light barrier 111 may cause a path of optical radiation emitted by the emitter 605 to travel through the tissue of the user 505 along a longer path before reaching the detector 603 at least because radiation reaching the detector 603 would have to travel over the light barrier 111 thus penetrating deeper into the tissue of the user 505. Deeper tissue penetration (e.g., longer optical path length) can ensure that optical radiation interacts with a greater amount of blood which can increase the accuracy of physiological measurements. The light barrier 111 may be formed of an opaque material configured to inhibit, reduce, and/or block the transmission of optical radiation therethrough. As shown, the light barrier 111 can deform the tissue of the user 505 because the light barrier 111 is raised from the surface 102 and protrudes toward the user 505.

FIG. 6C is a cross section view of the sensor module 100 taken along the line illustrated in FIG. 1A. The emitter chamber 105 can house one or more emitters 605, such as emitter 605a and emitter 605b as shown in this example. The emitters 605 can each emit optical radiation at a distinct wavelength. For example, the emitter 605a can emit optical radiation having a red color which may have a wavelength between 600 nm and 900 nm, between 600 nm and 800 nm, between 600 nm and 700 nm, about 620 nm, or about 660 nm. The emitter 605b can emit optical radiation in the infrared portion of the spectrum which have a wavelength between 900 nm and 100 nm, between 900 nm and 950 nm, between 950 nm and 1000 nm, or about 905 nm, or about 970 nm. In some implementations, the emitter chamber 105 can house additional emitters which emit radiation at other wavelengths such as between 500 nm and 600 nm, which may have a green color. In some implementations, the emitters 605 can emit optical radiation having a red color at two different wavelengths, such as 620 nm and 660 nm. In some implementations, the emitters 605 can emit optical radiation within the infrared portion of the spectrum at two different wavelengths, such as 905 nm and 970 nm. The emitter 605a may be a same distance from the detector 603 as the emitter 605b. In some implementations, emitter 605a and emitter 605b may each be a different distance from the detector 603.

The light barrier 111 can be curved. For example, a portion of the light barrier 111 may have a uniform width represented by D11 which adheres to a curvature of the surface 102. The ends of the light barrier 111 may be tapered. In some implementations, the width D1 of the light barrier 111 may vary along the length of the light barrier 111.

FIG. 6D is a cross section view of the sensor module 200 taken along the line illustrated in FIG. 2A. At least a portion of the electrode can define a portion of the surface 202. The surface 202 may have a same curvature as the surface 102 of sensor module 100. D15 can be a same length as D14. D15 can extend to a point on the surface 202 occupied by the electrode 201. Thus, the point of the surface 102 furthest from the level defined by the perimeter 104 may be located on the electrode 201. The sensor module 200 may be sized and/or shaped such that a finger of a user may contact the electrode 201 when their finger is placed on the sensor module 200.

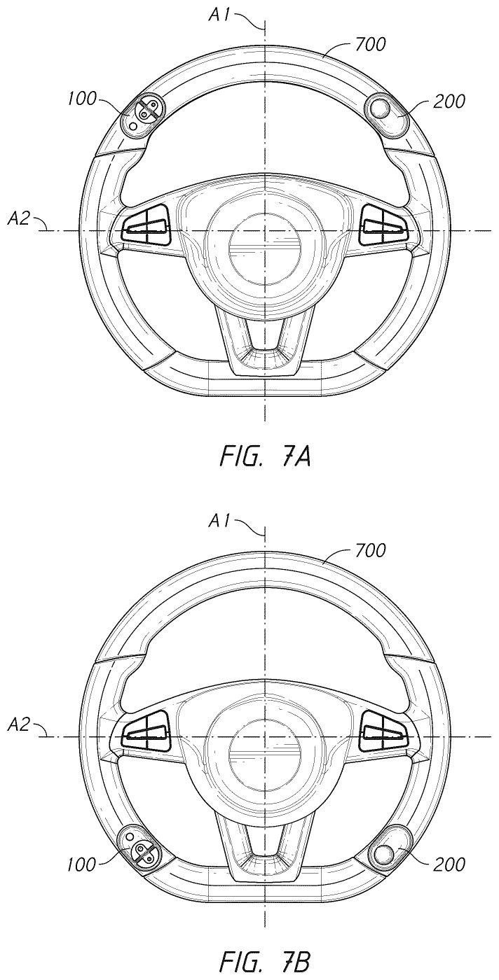

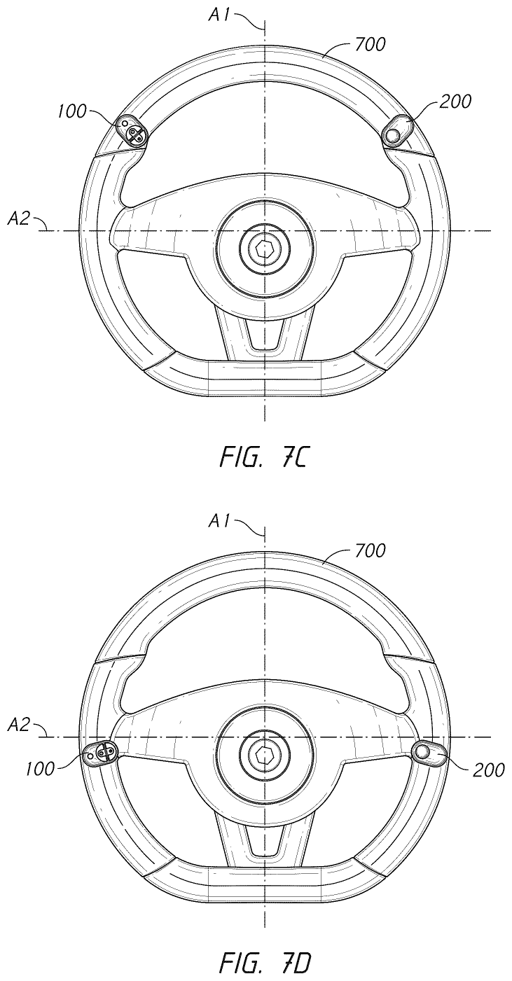

FIG. 7A illustrates an example implementation of physiological sensor module 100 and physiological sensor module 200 positioned on a front side of steering apparatus 700. Steering apparatus 700 may be circular and may be referred to as a steering wheel. In some implementations, steering apparatus 700 may be non-circular, such as rectangular. In some implementations, steering apparatus 700 may form an open loop and/or non-closed loop. For example, steering apparatus 700 may be a steering yoke which can be used to control an airplane. In some implementations, steering apparatus 700 may be a joystick such as may be used to control an airplane and/or helicopter. Axes A1 and A2 are superimposed on the image and illustrate, respectively, vertical and horizontal midlines of the steering apparatus 700. The sensor modules 100/200 may be positioned on and/or at least partially embedded within the steering apparatus 700. The sensor modules 100/200 may form a single integrated unit with the steering apparatus 700. A user, such as an operator of a vehicle, may place their hands on the steering apparatus 700, such as to drive the vehicle. As the user contacts the steering apparatus 700, the user may also contact the sensor modules 100 and/or 200. The user may contact the sensor module 100 and/or the sensor module 200 with a digit of their hand such as a finger or thumb. One hand of the user may contact the sensor module 100 and the other hand of the user may contact the sensor module 200, which may occur simultaneously or sequentially.

Sensor modules 100/200 may be positioned to the left of axis A1 and sensor module 200 can be positioned to the right of axis A1. Because sensor modules 100/200 are positioned on either side of axis A1, one hand a user may contact the sensor module 100 while the other hand contacts the sensor module 200. The sensor modules 100/200 may thus receive electrical signals conducted through opposite sides of the user's body which may improve ECG analysis of cardiac condition at least because proper ECG analysis relies on electrical signals from the right and left side of the heart (e.g., conducted through right and left hands). Sensor modules 100/200 may be positioned at a top portion of the steering apparatus 700, such as above axis A2. Sensor module 100 may be positioned within a same region of the steering apparatus 700 as the sensor module 200 (e.g., both on a top portion). In some implementations, one of sensor module 100/200 may be positioned on a top portion while the other is positioned on a bottom portion. Sensor module 100 and/or sensor module 200 may be positioned such that a length of the sensor module extends parallel and/or tangential to a perimeter defined by the steering apparatus 700. The shape of sensor module 100 and/or its position on steering apparatus 700 may be a mirror image of the shape and/or position of sensor module 200 about axis A1. In some implementations, that may not be the case.

FIG. 7B illustrates another example implementation of sensor modules 100/200 positioned steering apparatus 700. This example implementation illustrates sensor modules 100/200 positioned on a bottom portion of the steering apparatus 700 below axis A2. As shown, one or both of sensor modules 100/200 can be positioned on a bottom portion and one or both of sensor modules 100/200 can be positioned on a top portion.

FIG. 7C illustrates another example implementation of sensor modules 100/200 positioned on a back side of steering apparatus 700. As shown, sensor modules 100/200 may be positioned on the steering apparatus 700 such that a length of the sensor modules 100/200 extends perpendicular to the perimeter defined by the steering apparatus 700. As a user places their hands on the steering apparatus 700, the user's fingers may wrap around the steering apparatus 700 to the back side of the steering apparatus 700 and may contact the sensor modules 100/200.

FIG. 7D illustrates another example implementation of sensor modules 100/200 positioned on a back side of steering apparatus 700. As shown, the sensor modules 100/200 can be positioned below the axis A2. In some implementations, one or both of sensor modules 100/200 can be positioned on axis A2 and/or axis A1.

Various implementations are contemplated as combinations or rearrangements of the implementations such shown and/or described in FIGS. 7A-7D Advantageously, a user may contact a physiological sensor while operating a vehicle without having to remove their hands from a steering apparatus. In some implementations, any of the example sensor modules shown and/or described herein may be positioned at various locations on a vehicle other than the steering wheel. For example, one or more sensor modules may be positioned on a door of a vehicle, on a door handle, on a center console, on a dashboard, on an arm rest, on a gear shifter, or the like. In some implementations, one or more sensor modules may be positioned adjacent to a passenger in a vehicle and may collect physiological data from the passenger (e.g., a non-operating user of a vehicle).

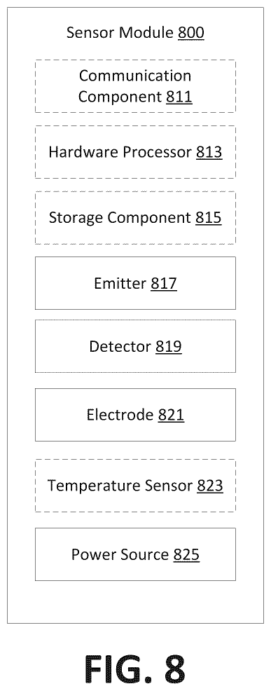

FIG. 8 is a block diagram of an example implementation of a sensor module 800 which may include similar structural and/or operational features as any of the other example sensor modules shown and/or described herein, such as sensor modules 100/200.

The sensor module 800 can optionally include a communication component 811. The communication component 811 can facilitate communication (via wireless, wired, and/or wire-like connection) between the sensor module 800 (and/or components thereof) and separate devices, such as separate sensors, systems, user devices, servers, or the like. For example, the communication component 811 can be configured to allow the sensor module 800 to wirelessly communicate with other devices, systems, and/or networks over any of a variety of communication protocols, including near-field communication protocols and far-field communication protocols. Near-field communication protocols, which may also be referred to as non-radiative communication, can implement inductive coupling between coils of wire to transfer energy via magnetic fields (e.g., NFMI). Near-field communication protocols can implement capacitive coupling between conductive electrodes to transfer energy via electric fields. Far-field communication protocols, which may also be referred to as radiative communication, can transfer energy via electromagnetic radiation (e.g., radio waves). The communication component 811 can communicate via any variety of communication protocols such as Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like. In some implementations, communication component 811 can implement human body communication (HBC) which can include capacitively coupling a transmitter and receiver via an electric field propagating through the human body. The communication component 811 can allow data and/or instructions to be transmitted and/or received to and/or from the sensor module 800 and separate computing devices. The communication component 811 can be configured to transmit and/or receive (for example, wirelessly) processed and/or unprocessed physiological data with separate computing devices. In some implementations, communication component 811 can transfer power required for operation of a computing device. The communication component 811 can be embodied in one or more components that are in communication with each other. The communication component 811 can include one or more of: transceivers, antennas, transponders, radios, emitters, detectors, coils of wire (e.g., for inductive coupling), and/or electrodes (e.g., for capacitive coupling). The communication component 811 can include one or more integrated circuits, chips, controllers, processors, or the like, such as a Wi-Fi chip and/or a Bluetooth chip.

The sensor module 800 can optionally include a hardware processor 813. The hardware processor 813 can comprise one or more integrated circuits. The hardware processor 813 can comprise and/or have access to memory. The hardware processor 813 can comprise and/or be embodied as one or more chips, controllers such as microcontrollers (MCUs), and/or microprocessors (MPUs). The hardware processor 813 can comprise a central processing unit (CPU). In some implementations, the hardware processor 813 can be embodied as a system-on-a-chip (SoC). The hardware processor 813 can be configured to implement an operating system which can allow multiple processes to execute simultaneously. The hardware processor 813 can be configured to execute program instructions to cause the sensor module 800 to perform one or more operations. The hardware processor 813 can be configured, among other things, to process data, execute instructions to perform one or more functions, and/or control the operation of the sensor module 800 or components thereof. For example, the hardware processor 813 can process physiological data obtained from physiological sensors and can execute instructions to perform functions related to storing and/or transmitting such physiological data.

The hardware processor 813 can access and process physiological data originating from the electrode 821. For example, the hardware processor 813 can implement electrocardiography processing techniques on voltage data originating from electrodes to analyze the user's cardiac activity. For example, the hardware processor 813 can generate ECG waveform data from electrode voltage data. An ECG waveform can include a trend line in the time domain having peaks and valleys with various voltage amplitude representing cardiac electrical activity. The hardware processor 813 can analyze features of the ECG waveform to determine cardiac characteristics, such as heart rate and/or arrythmias. ECG waveform characteristics can include peak amplitudes, valleys amplitudes, RR intervals, PR interval, QRS interval, QT interval, or the like.

The hardware processor 813 can access and process impedance data originating from electrodes on the user. The hardware processor 813 can generate impedance waveform data (in the time domain and/or frequency domain) from the impedance data. The hardware processor 813 can determine one or more physiological characteristics from the impedance data such as respiration rate, respiration pressure, and respiration volume.

The hardware processor 813 can access and process photoplethysmography (PPG) data originating from a PPG sensor such as an oximeter. The hardware processor 813 can determine characteristics of pulsatile blood flow from the PPG data. For example, the hardware processor 813 can determine volumetric variations in blood circulation from the PPG data and derive one or more parameters therefrom, such as blood oxygen saturation, hydration, hemoglobin content, pulse rate, blood pressure, respiration rate, respiration volume, cardiac output, perfusion index, pleth variability index, PPG waveform data, etc.

The hardware processor 813 can access and process temperature data originating from the temperature sensor 823. The hardware processor 813 can determine a subject's surface skin temperature and/or core body temperature from the temperature data.

The sensor module 800 can optionally include a storage component 815. The storage component 815 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. The storage component 815 can store data including processed and/or unprocessed physiological data obtained from physiological sensors, and/or communication data including device addresses of sensors and/or other monitoring hubs, for example. The storage component 815 can store program instructions that when executed by the hardware processor 813 cause the sensor module 800 to perform one or more operations.

The sensor module 800 can include one or more optical emitters 817 configured to emit optical radiation at one or more wavelengths, which can include visible light (e.g., green, orange, red), infrared and near-infrared. For example, the emitters 817 can emit radiation at one or more wavelengths between 500 nm and 1000 nm. The emitters 817 can include light emitting diodes (LEDs). In some implementations, the emitters 817 can emit the same wavelength from two separate emitters.

The sensor module 800 can include one or more optical detectors 819 configured to detect optical radiation emitted by the emitters 817 and attenuated by the tissue of a subject. The detector 819 can generate PPG data useable to determine pulsatile characteristics of the subject such as blood oxygen saturation, hydration, hemoglobin content, etc. The detector 819 and emitter 817 can operate together as an oximeter.

The sensor module 800 can include one or more electrodes, configured to measure electrical activity of the subject, such as cardiac signals.

The sensor module 800 can optionally include a temperature sensor 823 which can generate temperature data useable to determine a subject's temperature. The temperature sensor 823 can be a contact sensor or non-contact sensor. The temperature sensor 823 can be a thermistor, infrared sensor, thermocouple, and/or resistive sensor.

The sensor module 800 can include a power source 825. The power source 825 can provide power for components of the sensor module 800. The power source 825 can include a battery and/or capacitor. In some implementations, the power source 825 may be external to the sensor module 800. For example, the sensor module 800 can include or can be configured to connect to a cable which can itself connect to an external power source to provide power to the sensor module 800.

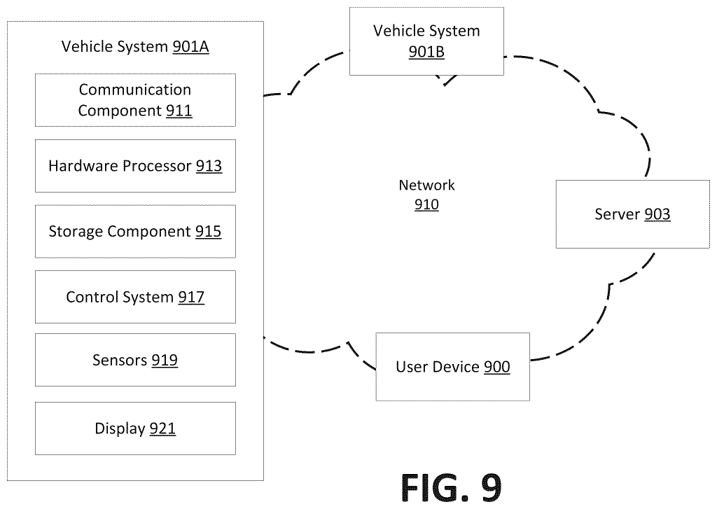

FIG. 9 is a schematic diagram of an example implementation of a vehicle system 901A in communication with one or more remote devices via network 910. The network 910 can include one or more communications networks. The network 910 can include a plurality of computing devices configured to communicate with one another. The network 910 can include routers. The network 910 can include the Internet. The network 910 can include a cellular network. The network 910 can include any combination of a body area network (e.g., implementing human body communication with capacitive coupling via the tissue of a user's body), a local area network (“LAN”) and/or a wide area network (“WAN”), or the like. Accordingly, various components or devices can communicate with one another directly or indirectly via any appropriate communications links and/or networks, such as network 910 (e.g., one or more communications links, one or more computer networks, one or more wired or wireless connections, the Internet, any combination of the foregoing, and/or the like).

Communication over the network 910 can include a variety of communication protocols, including wired communication, wireless communication, wire-like communication, near-field communication (such as inductive coupling between coils of wire or capacitive coupling between conductive electrodes), and far-field communication (such as transferring energy via electromagnetic radiation (e.g., radio waves)). Example communication protocols can include Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like.

The server 903 may comprise one or more computing devices including one or more hardware processors. The server 903 may comprise program instructions configured to cause the server 903 to perform one or more operations when executed by the hardware processors. The server(s) 106 can include, and/or have access to (e.g., be in communication with and/or host) a storage device, database, or system which can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. In some implementations, the server(s) 106 can include and/or be in communication with a hosted storage environment that includes a collection of physical data storage devices that may be remotely accessible and may be rapidly provisioned as needed (commonly referred to as “cloud” storage). Data stored in and/or accessible by the server(s) 106 can include physiological data including historical physiological data previously obtained by the one or more sensors 102 and/or communication data including, for example, device addresses of monitoring hubs, sensors, or the like. In some implementations, the server(s) 106 can comprise and/or be in communication with an electronic medical records (EMR). An EMR can comprise a propriety EMR. An EMR can comprise an EMR associated with a hospital. An EMR can store data including medical records. In some implementations, the server 903 can include a vehicle related system or service that can provide vehicular communications, security, emergency services, navigation, and/or diagnostics.

The vehicle system 901B can include similar structural and/or operational features as vehicle system 901A as shown and/or described herein. The vehicle system 901A may be associated with one vehicle, and vehicle system 901B may be associated with another vehicle. In some implementations, more than two vehicle systems (each associated with a separate vehicle) may be in communication via network 910. Each of the vehicle systems (e.g., 901A, 901B) in communication via network 910 may be associated with a same user, such as owned by a same user and/or authorized by the user to communicate with each other. Thus, a user's information may be shared across multiple vehicles if a user uses more than one vehicle. The vehicle system 901 may communicate data, such as physiological data, to the user device 900, the vehicle system 901B, and/or to the server 903. Advantageously, multiple vehicles may access and/or process physiological data obtained from the user by different vehicles. For example, vehicle system 901B may access data originating from vehicle system 901A, and vice versa.

The user device 900 can include one or more hardware processors and/or program instructions configured to cause the user device 900 to perform one or more operations when executed by the hardware processors. The user device 900 may be one or more of a computer, a laptop, a tablet, or a phone. The user device 900 may be a wearable device such as a smartwatch or an auricular device such as an earbud. The user device 900 may be a medical device such as an insulin pump. The user device 900 can include one or more physiological sensors configured to generate physiological data of a user. For example, the user device 900 can include one or more of acoustic sensors, optical sensors, motion sensors, temperatures sensors, electrical sensors, voltage sensors, impedance sensors, etc. The user device 900 can include an oximeter. The user device 900 can include a photoplethysmography (PPG) sensor configured to measure volumetric variation in blood circulation and derive one or more parameters therefrom, such as pulse rate, blood oxygen saturation, respiration rate, etc. The user device 900 can include one or more optical emitters configured to emit optical radiation of a plurality of wavelengths, which may include visible light. The user device 900 can include one or more optical detectors configured to detect optical radiation attenuated by the tissue of subject (which may have been emitted by optical emitters) and generate data relating to the pulsatile characteristics of the subject. The user device 900 can include electrocardiogram (ECG) sensors, including one or more electrodes, configured to measure electrical activity of the subject, such as cardiac signals. The user device 900 can include electroencephalography (EEG) sensors. The user device 900 can measure and/or generate data relating to respiration rate, blood oxygen saturation (e.g., SpO2), heart rate, pulse rate, skin temperature, core body temperature, orientation, or the like. The user device 900 may be associated with (e.g., worn by and collecting physiological data of) a user of a vehicle such as an operator of a vehicle, a driver of a vehicle, or a passenger of a vehicle.

The vehicle system 901A can include a communication component 911, one or more hardware processors 913, a storage component 915, one or more control system 917, one or more sensors 919, and a display 921.

The communication component 911 can facilitate communication (via wireless, wired, and/or wire-like connection) between the vehicle system 901A (and/or components thereof) and separate devices, such as separate monitoring hubs, monitoring devices, sensors, systems, servers, or the like. For example, the communication component 911 can be configured to allow the vehicle system 901A to wirelessly communicate with other devices, systems, and/or networks over any of a variety of communication protocols, including near-field communication protocols and far-field communication protocols. Near-field communication protocols, which may also be referred to as non-radiative communication, can implement inductive coupling between coils of wire to transfer energy via magnetic fields (e.g., NFMI). Near-field communication protocols can implement capacitive coupling between conductive electrodes to transfer energy via electric fields. Far-field communication protocols, which may also be referred to as radiative communication, can transfer energy via electromagnetic radiation (e.g., radio waves). The communication component 911 can communicate via any variety of communication protocols such as Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like. In some implementations, communication component 911 can implement human body communication (HBC) which may include capacitively coupling a transmitter and receiver via an electric field propagating through the human body. The communication component 911 can allow data and/or instructions to be transmitted and/or received to and/or from the vehicle system 901A and separate computing devices. The communication component 911 can be configured to transmit and/or receive (for example, wirelessly) processed and/or unprocessed physiological data with separate computing devices including physiological sensors, other monitoring hubs, remote servers, or the like. In some implementations, communication component 911 can transfer power required for operation of a computing device. The communication component 911 can be embodied in one or more components that are in communication with each other. The communication component 911 can include one or more of: transceivers, antennas, transponders, radios, emitters, detectors, coils of wire (e.g., for inductive coupling), and/or electrodes (e.g., for capacitive coupling). The communication component 207 can include one or more integrated circuits, chips, controllers, processors, or the like, such as a Wi-Fi chip and/or a Bluetooth chip.

The hardware processor 913 can comprise one or more integrated circuits. The hardware processor 913 can comprise and/or have access to memory. The hardware processor 913 can comprise and/or be embodied as one or more chips, controllers such as microcontrollers (MCUs), and/or microprocessors (MPUs). The hardware processor 913 can comprise a central processing unit (CPU). In some implementations, the hardware processor 913 can be embodied as a system-on-a-chip (SoC). The hardware processor 913 can be configured to implement an operating system which can allow multiple processes to execute simultaneously. The hardware processor 913 can be configured to execute program instructions to cause the vehicle system 901A (or other devices in communication with vehicle system 901A) to perform one or more operations. The hardware processor 913 can be configured, among other things, to process data, execute instructions to perform one or more functions, and/or control the operation of the vehicle system 901A or components thereof. For example, the hardware processor 913 can process physiological data obtained from physiological sensors and can execute instructions to perform functions related to storing and/or transmitting such physiological data. In some implementations, the hardware processor 913 may be remote the vehicle system 901A. The hardware processor 913 may be referred to as a controller.

The hardware processor 913 can access and process physiological data originating from the sensors 919. For example, the hardware processor 913 can implement electrocardiography processing techniques on voltage data originating from electrodes to analyze the user's cardiac activity. For example, the hardware processor 913 can generate ECG waveform data from electrode voltage data. An ECG waveform can include a trend line in the time domain having peaks and valleys with various voltage amplitude representing cardiac electrical activity. The hardware processor 813 can analyze features of the ECG waveform to determine cardiac characteristics, such as heart rate and/or arrythmias. ECG waveform characteristics can include peak amplitudes, valleys amplitudes, RR intervals, PR interval, QRS interval, QT interval, or the like.

The hardware processor 913 can access and process impedance data originating from sensors 919. The hardware processor 913 can generate impedance waveform data (in the time domain and/or frequency domain) from the impedance data. The hardware processor 913 can determine one or more physiological characteristics from the impedance data such as respiration rate, respiration pressure, and respiration volume.

The hardware processor 913 can access and process photoplethysmography (PPG) data originating from a PPG sensor such as an oximeter. The hardware processor 913 can determine characteristics of pulsatile blood flow from the PPG data. For example, the hardware processor 913 can determine volumetric variations in blood circulation from the PPG data and derive one or more parameters therefrom, such as blood oxygen saturation, hydration, hemoglobin content, pulse rate, blood pressure, respiration rate, respiration volume, cardiac output, perfusion index, pleth variability index, PPG waveform data, etc. In some implementations, the processor can determine and/or access pulse transit time and/or pulse arrival time from PPG data. In some implementations, the processor can determine a level of carbon monoxide in the user's blood from PPG data.

The hardware processor 913 can access and process temperature data originating from a temperature sensor. The hardware processor 913 can determine a subject's surface skin temperature and/or core body temperature from the temperature data.

The storage component 915 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. Such stored data can be processed and/or unprocessed physiological data obtained from physiological sensors, for example. The storage component 915 can store program instructions that when executed by the hardware processor 913 cause the vehicle system 901A to perform one or more operations.

The storage component 915 can store historical data such as physiological data received from the sensors 919, computing device, and/or server 903. A user may spend long periods of time throughout ordinary daily life operating a vehicle. Advantageously, the storage component 915 can accumulate large amounts of physiological data of the user without the user having to depart from their ordinary daily routine. The processor 913 can analyze historical data to generate a physiological profile for the user. The processor 913 can generate alerts or notifications for the user based on analyzing real-time and/or historical physiological data. The processor 913 can detect deviations from normal (e.g., a mean and/or median). The processor 913 can monitor trends in data. In some implementations, the processor 913 can advise a user to visit a healthcare provider based on analyzing physiological data of the user.

The control system 917 (which may also be referred to as a controller) can include and/or control one or more operations of the vehicle. For example, the control system 917 can control an audio emitted from a vehicle sound system, lighting conditions of a light system on an interior or exterior of the vehicle, temperature of a vehicle temperature system, vehicular climate within an interior of the vehicle, seat conditions, such as seat positions, orientations, seat massage operations, pedal positions, driving variabilities, or the like. As an additional example, the control system 917 can control driving operations of a vehicle autonomous driving system, such as a destination of where the vehicle is traveling, a speed of the vehicle, etc. As an additional example, the control system 917 can control one or more safety features of the vehicle such as whether the vehicle starts or turning the vehicle off, controlling a direction of the vehicle (e.g., to reduce unintentional and/or undesired swerving or veering out of a lane), or controlling a speed of the vehicle such as to ensure compliance with laws.

The sensors 919 can include similar structural and/or operational features as sensor module 100 or sensor module 200 shown and/or described herein. The sensors 919 can be positioned on any portion of the vehicle such as a door, a steering wheel, a center console, a dashboard, an arm rest, a control panel, or the like. The sensors 919 can include one or more physiological sensors configured to generate physiological data of physiological parameters. The sensors 919 can include acoustic sensors, optical sensors, motion sensors, temperatures sensors, electrical sensors, voltage sensors, impedance sensors, etc. The sensors 309 can include an oximeter configured to generate photoplethysmography (PPG) data useable to determine volumetric variations in blood circulation and derive one or more parameters therefrom, such as blood oxygen saturation, hydration, hemoglobin content, pulse rate, blood pressure, respiration rate, respiration volume, cardiac output, perfusion index, pleth variability index, PPG waveform data, etc. The sensors 919 can include one or more optical emitters configured to emit optical radiation of a plurality of wavelengths. The sensors 919 can include one or more optical detectors configured to detect optical radiation attenuated by the tissue of subject (which may have been emitted by optical emitters) and generate data relating to the pulsatile characteristics of the subject. The sensors 919 can include electrocardiogram (ECG) sensors, including one or more electrodes, configured to measure electrical activity of the subject, such as cardiac signals. The sensors 919 can include electroencephalography (EEG) sensors. The sensors 919 can measure and/or generate data relating to respiration rate, blood oxygen saturation (e.g., SpO2), heart rate, pulse rate, skin temperature, core body temperature, orientation, or the like.

The sensors 919 can continuously and/or periodically monitor a user's physiological data. The sensors 919 can continuously and/or periodically provide data to the vehicle system 901. The sensors 919 can collect physiological data of the user as the user operates the vehicle. The sensors 919 can periodically perform a spot check. The sensors 919 can check a user's physiological data as the user enters the vehicle, as the user turns the vehicle on, as the user exits the vehicle, and/or based on the passage of time.

The sensors 919 can include a camera configured to capture images of a user. The camera can be positioned on a dashboard of the vehicle, one or near a steering wheel, on a rear-view mirror, etc. The hardware processor 913 can perform image processing techniques (e.g., pattern recognition) on camera images to determine a physiological status of the user. For example, the processor 913 can process image data to determine a user's head position (which may indicate attention level such as if the user is not looking ahead at the road and/or drowsiness level if the user's head is facing downward), a user's distance from a steering wheel (which may indicate sleep status or unsafe seating conditions), perspiration on the user's skin, a user's eye status (e.g., eyelids are open or shut, pupils are dilated or constricted, pupil tracking to analyze eye movement which may indicate user's focus of attention or concentration level, or the like), a user's emotions, 3D gesturing from user's hands indicative of user's commands, respiration rate including respiration patterns which may indicate an alertness or drowsiness level. In some implementations, the sensors can include a radar sensor, infrared sensor, an ultra-wide band sensor, a mmWave sensor, etc. and the processor 913 can process data from the sensor to determine one or more of the conditions described above. In some implementations, the processors 913 can determine a user's physiological status, such as pulse rate, respiration rate, pulse rate variability, using remote photoplethysmography (PPG) with data from the sensors 919 (e.g., a light sensor, radar sensor, mmWave sensor, etc.).

The sensors 919 can collect and/or generate data relating to an operation of a vehicle. For example, the sensor 919 can generate position data to indicate of a location of the vehicle, or motion data to indicate an angular and/or linear acceleration or velocity of the vehicle. The sensor 919 can determine a proximity of the vehicle to other vehicles. The sensor 919 can determine whether the vehicle is within a lane or proximity to painted lines on the road. The sensor 919 can include one or more of an optical sensor, a light sensor, a camera, an infrared sensor, a radar sensor, a LIDAR sensor, a mmWave sensor, an ultra-wide band sensor, a temperature sensor, a GPS, an accelerometer, a gyroscope, or the like.

The vehicle system 901 can access data originating from the sensors 919 and/or user devices 900 and can process the data and/or store the data in storage component 915. The processor 913 can analyze real-time data (e.g., data as it originates from the sensors 919 and/or user devices 900) and/or historical data (e.g., data stored in the storage component 915). In some implementations, the processor 913 can receive one or more instructions from the server 903. For example, the server 903 can generate instructions to send to the processor 913 based on processing data originating from the sensors 919 and/or user devices 900. The processor 913 (and/or server) can determine a physiological status of a user of the vehicle based on data originating from sensors 919 and/or user devices 900. The hardware processor 913 can process physiological data originating from sensors 913 and/or user device 900 to determine a physiological status of a user or can receive an indication of a physiological status of the user (e.g., based on processed physiological data) from the user device 900 and/or server 903.

In some implementations, the server 903 can generate instructions to control the hardware processor 913 to control the vehicle system 901A and can communicate those instructions to the vehicle system 901A via the network 910. In some implementations, the user device 900 can generate instructions to control the hardware processor 913 to control the vehicle system 901A and can communicate those instructions to the vehicle system 901A via the network 910.

The hardware processor 913 can cause the control system 917 to adjust one or more operations of the vehicle based on data originating from the sensors 919 and/or user devices 900, such as physiological data. For example, the hardware processor 913 can cause the control system 917 to perform one or more safety operations based on data originating from the sensors 919 and/or user devices 900, such as physiological data. For example, the hardware processor 913 can cause the control system 917 to initiate a call to a healthcare provider or recommend to the user that they call their healthcare provider based on data originating from the sensors 919 and/or user devices 900. As an additional example, the hardware processor 913 can cause the control system 917 to provide recommendations to the user regarding their health (e.g., to visit a doctor) or generate alerts or notifications relating to the user's health based on data originating from the sensors 919 and/or user devices 900.

As an additional example, the hardware processor 913 can cause the control system 917 to initiate one or more autonomous driving features based on data originating from the sensors 919 and/or user devices 900. For example, the control system 917 can cause the vehicle to slow down, pull over to the side of the ride, cause the vehicle cease to operate, cause the vehicle to autonomously navigate to a healthcare facility (e.g., the nearest hospital), generate directions to a nearby healthcare facility, or the like based on a determination that a user is not capable of operating a vehicle or requires medical attention. A user may not be capable of operating a vehicle if they are drowsy, inebriated, stressed, experiencing significant physiological conditions such as cardiac arrest, cardiac arrythmias, low blood oxygen saturation, abnormal blood glucose levels, abnormal respiration rates, irregular eye movement, or the like. Drowsiness can be determined based on eye movement, eye open or closed status, respiration rate, heart rate, etc. Inebriation may be determined based on pupil dilation, respiration rate, heart rate, eye movement, eye open or shut status, blood oxygen saturation, blood alcohol content, or the like. In some implementations, the control system 917 can prevent a vehicle from starting (e.g., when attempted by a user) if a user is not capable of operating the vehicle as described above.

As an additional example, the hardware processor 913 can cause the control system 917 to control operation of airbags based on data originating from the sensors 919 and/or user devices 900. For example, the control system 917 can activate or deactivate an airbag from being deployed (e.g., in the case of a collision) based on a user's position relative to a steering wheel (which may be determined based on data originating from a camera, radar sensor, or the like). For example, if a user is too close to a steering wheel (as may be determined from camera image data or radar sensor data), deploying the airbag can cause serious harm to the user and thus the hardware processor 913 can cause the control system 917 to deactivate the airbag from deploying while the user, or body part of the user (e.g., head, arms, etc.) is within a proximity distance of the steering wheel. The control system 917 can activate the airbag system such that the airbags can deploy when a user is positioned safely such as with respect to the steering wheel.

The hardware processor 913 can cause the control system 917 to adjust one or more operations of the vehicle based on data originating from the sensors 919 and/or user devices 900, such as physiological data. For example, the hardware processor 913 can cause the control system 917 to adjust one or more environmental conditions within an interior region of the vehicle (e.g., the cabin) to increase a user's comfort level based on data originating from the sensors 919 and/or user devices 900, such as physiological data. As an example, the hardware processor 913 can access temperature data from the sensors 919 and/or user devices 900 indicating a temperature of a user of the vehicle and in response can cause the control system 917 to adjust a cabin temperature of the vehicle to facilitate a comfort of the user. As another example, the hardware processor 913 can cause the control system 917 to adjust a cabin temperature of the vehicle based on detecting sweat on the user's skin from processing camera image data (e.g., from a camera positioned on a dashboard or on or near a steering wheel and oriented toward a user such as a face of a user). As another example, the hardware processor 913 can cause the control system 917 to adjust an audio system (e.g., turning on or off, volume, radio station, soundtrack, playlist, etc.) based on a stress level and/or heart rate of the user based on data from the sensors 919 and/or user devices 900. As another example, the hardware processor 913 can cause the control system 917 to generate an alert in response to detecting a drowsiness of the user (e.g., based on heart rate, respiration rate, image data indicating head orientation of user, image data indicating eye open or closed status of user, etc.) based on data from the sensors 919 and/or user devices 900 to prevent the user from falling asleep while driving. As another example, the hardware processor 913 can cause the control system 917 to emit a particular scent into the cabin of the vehicle based on data originating from the sensors 919 and/or user devices 900. As another example, the hardware processor 913 can cause the control system 917 to control a seat (e.g., activating massage operations, adjusting seat position, etc.) based on data originating from the sensors 919 and/or user devices 900. For example, the control system 917 activate a massage based on detecting a stress level of the user or can adjust a seat position to a user's preset position based on detecting that particular user is in the seat. In some implementations, the control system 917 can adjust one or more positions such as seat positions, seat recline angle, seat height, steering wheel position, seat air cushion inflation, pedal position, etc. based on time and/or data from the sensors 919 and/or user devices 900. Periodically adjusting one or more of these positions based on at least time can improve a user's circulatory blood flow.

The hardware processor 913 can cause the control system 917 to perform user verification or authentication with data originating from the sensors 919 and/or user devices 900. For example, based on verifying an identify of the user, the control system 917 can allow/restrict a user to enter a vehicle (e.g., keyless entry), can allow a car to start or prevent the car from starting, can adjust the settings of the vehicle (e.g., audio volume, temperature, pedal position, seat position, steering wheel position, inflate seat air cushions, etc.). The hardware processor 913 can verify an identity of the user based on facial recognition, voice recognition, eye recognition (e.g., a retinal scan), fingerprint recognition, or other physiological data of the user including heart rate, respiration rate, blood oxygen saturation, weight of the user (as determined by a weight sensor in the seat of the vehicle for example). In some implementations, the hardware processor 913 can verify an identify of a user based on non-physiological data, such as data received from user device 900 including, for example, a device address (e.g., IP address, MAC address) of the user device 900 or other data indicating an identity of the user device 900 or user associated with the user device 900.

The hardware processor 913 can control an operation of the control system 917 based on a dynamic set of variables. For example, the hardware processor can control any of the example operations of the control system 917 as described herein based on data from the sensors 919 only, from the user device 900 only, or from the sensors 919 and the user device 900 in combination. The dynamic set of variables can change based on whether the data is available and/or whether the data is reliable. For example, the hardware processor 913 can access data from a sensor 919 disposed within the vehicle and from a user device 900 worn by the user to determine a certain parameter such as a user's competency to operate the vehicle. However, in some instances, the hardware processor 913 can continue to determine the same parameter (e.g., user's competency) even when data from the user device 900 is not available such as if the user is not currently wearing the user device 900 and/or if the data from the user device 900 is not reliable (e.g., has a low confidence value).

The display 921 may be a vehicle dashboard display. The display 921 can include an LED screen, an LCD screen, an OLED screen, a QLED screen, a plasma display screen, a quantum dot display screen, or the like. The display 921 can be responsive to touch. For example, the display screen may comprise a touchscreen such as a resistive touchscreen, a capacitive touchscreen, an infrared touchscreen, a surface acoustic wave touchscreen, or the like. The processor 913 can cause the display 921 to display indicia of one or more physiological conditions, including physiological parameters, to the user of the vehicle. The processor 913 can generate user interface data to cause the display 921 to render one or more user interfaces. The display 921 can display indicia of physiological data originating from sensors 919 and/or indicia of physiological data originating from user device 900. In some implementations, the display 921 can display non-physiological related data such as data relating to an operation of a vehicle such as vehicle speed, location, etc.

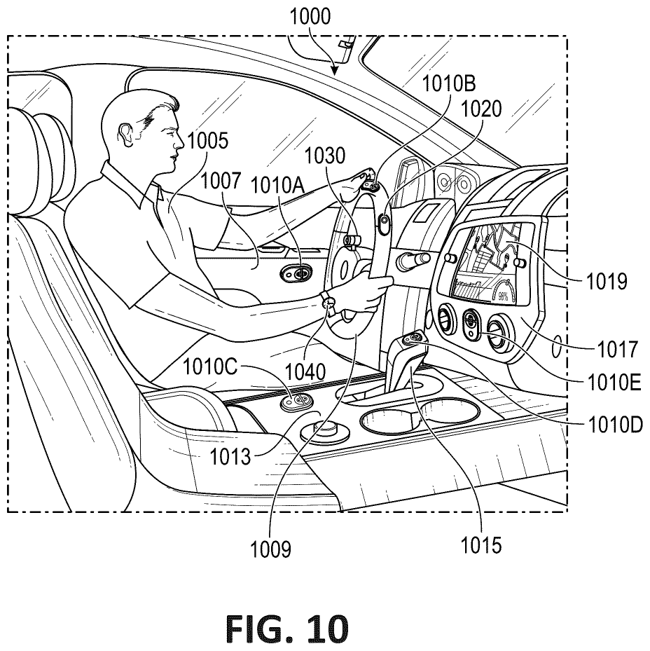

FIG. 10 illustrates an example implementation of a vehicle 1000 comprising one or more sensors, such as sensors 1010A-1010E, sensor 1020, and/or sensor 1030. The sensors 1010A-1010E can include similar structural and/or operational features as sensor module 100 shown and/or described herein. Any of sensors 1010A-1010E can comprise a pulse oximeter, for example emitters configured to emit optical radiation and detectors configured to detect optical radiation which can be used to determine physiological parameters including blood oxygen saturation and/or pulse rate. Any of sensors 1010A-1010E can comprise a temperature sensor. Any of sensors 1010A-1010E can comprise one or more electrodes such as for performing electrocardiography. Sensor 1020 can include similar structural and/or operational features as sensor module 200 shown and/or described herein. Sensor 1020 can comprise one or more electrodes such as for performing electrocardiogram some implementations, one or more of sensors 1010A-1010E and/or sensor 1020 can include one or more hardware processors for processing physiological data. In some implementations, the vehicle 1000 can include one or more hardware processors (separate from the sensors) configured to access and/or process physiological data originating from any of the sensors disposed within the vehicle. In some implementations, one or more hardware processors remote to the vehicle 1000 can access and/or process physiological data originating from any of the sensors disposed within the vehicle 1000.

Sensor 1010A can be positioned on a door 1007 of the vehicle. In some implementations, the sensor 1010A can be positioned on a handle of the door 1007. The user 1005 can place a finger (e.g., from their left hand) on the sensor 1010A for a physiological measurement. The user 1005 can place their finger on the sensor 1010A while they are driving, while the vehicle 1000 is stationary, while they are entering or exiting the vehicle, etc.

Sensor 1010B can be positioned on the steering wheel 1009. The sensor 1010B can be positioned at any location on the steering wheel 1009 such as at a top center of the steering wheel 1009. The user 1005 can place a finger (e.g., from their left hand) on the sensor 1010B for a physiological measurement. The user 1005 can place their finger on the sensor 1010B while they are driving, while the vehicle 1000 is stationary, etc.

Sensor 1020 can be positioned on the steering wheel 1009. The sensor 1010B can be positioned at any location on the steering wheel 1009 such as on a right side of the steering wheel 1009. The user 1005 can place a finger (e.g., from their right hand) on the sensor 1020 for a physiological measurement. The user 1005 can place their finger on the sensor 1020 while they are driving, while the vehicle 1000 is stationary, etc. In some implementations, the user 1005 may place a finger from their right hand on sensor 1020 while placing a finger from their left hand on sensor 1010B, for example, to implement an ECG measurement (which may require contact with multiple portions of the body of the user 1005). Advantageously, the user can implement a physiological measurement, such as ECG, while they are operating the vehicle 1005 without removing their hands from the steering wheel 1009.

Sensor 1010C can be positioned on a console 1013. The user 1005 can place a finger (e.g., from their right hand) on the sensor 1010C for a physiological measurement. The user 1005 can place their finger on the sensor 1010C while they are driving, while the vehicle 1000 is stationary, etc.

Sensor 1010D can be positioned on a gear selector 1015. The user 1005 can place a finger (e.g., from their right hand) on the sensor 1010D for a physiological measurement. The user 1005 can place their finger on the sensor 1010D while they are driving, while the vehicle 1000 is stationary, while they are changing gears, etc.

Sensor 1010E can be positioned on a dashboard 1017. The user 1005 can place a finger (e.g., from their right hand) on the sensor 1010E for a physiological measurement. The user 1005 can place their finger on the sensor 1010E while they are driving, while the vehicle 1000 is stationary, etc.

The quantity of sensors 1010A-1010E, 1020 and/or their positions in the vehicle 1000 as shown in FIG. 10 are provided as an example. In some implementations, the vehicle 1000 can include more sensors or fewer sensors. In some implementations, the sensors can be positioned at different locations in the vehicle than what is shown.

In some implementations, the user 1005 may have a user device 1040 such as a phone or a wearable device. The user device 1040 may be a watch. The user device 1040 may comprise one or more sensors configured to perform physiological measurements and generate physiological data. The vehicle 1000 (or hardware processor thereof) can access physiological data originating from the user device 1040. The vehicle 1000 can perform one or more operations based on data originating from the user device 1040, such as adjusting settings within the cabin of the vehicle 1000 or adjusting a driving operation of the vehicle or implementing autonomous driving features. In some implementations, the vehicle 1000 can combine physiological data originating from the user device 1040 with physiological data originating from any of sensors 1010A-1010E or sensor 1020, such as to refine physiological parameters or to generate combined physiological parameters.

A sensor 1030 can be positioned within the car, such as on the steering wheel 1009. The sensor 1030 can be configured to detect electromagnetic radiation, including radio waves, microwaves, infrared, visible light, and/or ultraviolet. The sensor 1030 can comprise a radar sensor configured to detect radio waves and/or microwaves such as an ultra-wide band (UWB) sensor or a mmWave sensor. The sensor 1030 can be configured to detect infrared indicative of thermal energy. The sensor 1030 can be configured to detect visible light. The sensor 1030 can comprise a camera such as a single lens camera or multi-lens camera (e.g., 3D, depth, stereovision). The sensor 1030 can comprise a proximity sensor. The sensor 1030 can be oriented toward the user 1005 and can capture images of the user 1005 and/or electromagnetic radiation emanating from a direction of the user 1005, such as infrared energy which may indicate the user's body temperature. The sensor 1030 can implement remote PPG to determine physiological parameters of the user 1005. For example, the sensor 1030 can capture images of the user's face to determine volumetric changes in the facial tissues of the user 1005. The sensor 1030 can determine a proximity of the user 1005 to the steering wheel 1009. The sensor 1030 can verify an identity of the user 1005 such as by capturing images of the user's face and implementing facial recognition.

A display 1019 can be positioned on the dashboard 1017. The display 1019 can include an LED screen, an LCD screen, an OLED screen, a QLED screen, a plasma display screen, a quantum dot display screen, or the like. The display 1019 can be responsive to touch. For example, the display screen can comprise a touchscreen such as a resistive touchscreen, a capacitive touchscreen, an infrared touchscreen, a surface acoustic wave touchscreen, or the like. The display 1019 can display physiological data, such as parameters from data originating from sensors positioned within the vehicle and/or worn by the user. The display 1019 can display physiological trends, charts, graphs, gauges, dials, numbers, or the like. The display 1019 can display information relating to an operation of the vehicle. For example, the display 1019 can display a map directing a user to a destination. In some implementations, the display 1019 can automatically display information based on physiological data of the user 1005. For example, the display 1019 can automatically display a route on a map to the nearest healthcare facility based on the physiological parameters of the user 1005 exceeding a threshold.

### Additional Considerations

Certain categories of persons, such as caregivers, clinicians, doctors, nurses, and friends and family of a user, may be used interchangeably to describe a person providing care to the user. Furthermore, patients or users used herein interchangeably refer to a person who is wearing a sensor or is connected to a sensor or whose measurements are used to determine a physiological parameter or a condition. Parameters may be, be associated with, and/or be represented by, measured values, display icons, alphanumeric characters, graphs, gages, power bars, trends, or combinations. Real time data may correspond to active monitoring of a user, however, such real time data may not be synchronous to an actual physiological state at a particular moment. Measurement value(s) of a parameter and the parameter used herein such as, SpO2, RR, PaO2 and the like, unless specifically stated otherwise, or otherwise understood with the context as used is generally intended to convey a measurement or determination that is responsive to the physiological parameter.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable. The various features and processes described herein may be used independently of one another, or may be combined in various ways. For example, elements may be added to, removed from, or rearranged compared to the disclosed example implementations. All possible combinations and sub-combinations are intended to fall within the scope of this disclosure.

Any methods and processes described herein are not limited to any particular sequence, and the blocks or states relating thereto can be performed in other sequences that are appropriate. For example, described blocks or states may be performed in an order other than that specifically disclosed, or multiple blocks or states may be combined in a single block or state, or certain method or process blocks may be omitted, or certain blocks or states may be performed in a reverse order from what is shown and/or described. The example blocks or states may be performed in serial, in parallel, or in some other manner. Blocks or states may be added to or removed from the disclosed example implementations.

The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct entities or other users. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames).

Many other variations than those described herein will be apparent from this disclosure. For example, depending on the implementation, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

Various illustrative logical blocks, modules, routines, and algorithm steps that may be described in connection with the disclosure herein can be implemented as electronic hardware (e.g., ASICs or FPGA devices), computer software that runs on computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, controller, microcontroller, or state machine, combinations of the same, or the like. A processor can include electrical circuitry configured to process computer-executable instructions. A processor can include an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

As used herein, “real-time” or “substantial real-time” may refer to events (e.g., receiving, processing, transmitting, displaying etc.) that occur at a same time as each other, during a same time as each other, or overlap in time with each other. “Real-time” may refer to events that occur at distinct or non-overlapping times the difference between which is imperceptible and/or inconsequential to humans such as delays arising from electrical conduction or transmission. A human may perceive real-time events as occurring simultaneously, regardless of whether the real-time events occur at an exact same time. As a non-limiting example, “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, “system,” “instrument,” “apparatus,” and “device” generally encompass both the hardware (for example, mechanical and electronic) and, in some implementations, associated software (for example, specialized computer programs for operational control) components.

It should be emphasized that many variations and modifications may be made to the herein-described implementations, the elements of which are to be understood as being among other acceptable examples. All such modifications and variations are intended to be included herein within the scope of this disclosure. Any section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A steering apparatus configured for physiological monitoring, the steering apparatus comprising:
a left portion and a right portion configured to rotate about a central portion of the steering apparatus to control the vehicle, the left portion being symmetrical with the right portion across the central portion,
wherein the left portion comprises a depressed region forming a curved surface that is shaped to receive a digit of a left hand of a user when the user places their left hand on the steering apparatus, the curved surface being elongate extending along a length of the left portion, wherein a first sensor is positioned within the curved surface,
wherein the right portion comprises another depressed region forming another curved surface on the right portion that is shaped to receive a digit of a right hand of the user when the user places their right hand on the steering apparatus, the other curved surface extending along a length of the right portion, wherein a second sensor is positioned within the another curved surface.

2. The steering apparatus of claim 1 wherein a controller is configured to control an operation on the vehicle based on the physiological characteristics of the user determined from sensor data from the first sensor or the second sensor.

3. The steering apparatus of claim 1 wherein the curved surface comprises a left arm (LA) electrode configured to contact the digit of the left hand and respond to electrical voltages conducted through the left hand of the user to the LA electrode, wherein the other curved surface comprises a right arm (RA) electrode configured to contact the digit of the right hand of the user and respond to electrical voltages conducted through the right hand to the RA electrode, the RA electrode being operably coupled with the LA electrode to form an electrode pair, the RA electrode and the LA electrode being physically and electrically isolated from each other on the steering apparatus.

4. The steering apparatus of claim 1 further comprising an oximeter positioned with the depressed region of the left portion, the oximeter comprising one or more optical emitters configured to emit optical radiation away from the curved surface toward the digit of the left hand of the user, the oximeter comprising one or more optical detectors configured to generate plethysmography data responsive to detecting optical radiation attenuated by the tissue of the user.

5. The steering apparatus of claim 4 further comprising:
an emitter chamber embedded within the curved surface housing the one or more optical emitters;
a detector chamber embedded within the curved surface housing the one or more optical detectors; and
a light barrier positioned on the curved surface between the emitter chamber and the detector chamber, wherein the light barrier extends from the curved surface and is configured to:
induce optical radiation emitted from the one or more optical emitters to penetrate the digit of the user before arriving at the optical detector; and
inhibit optical radiation emitted from the one or more optical emitters from travelling within a gap between the curved surface and the digit of the left hand of the user.

6. The steering apparatus of claim 4 wherein the LA electrode at least partially surrounds the one or more emitters and/or the one or more detectors.

7. The steering apparatus of claim 1 further comprising a second left arm (LA) electrode positioned on the curved surface of the depressed region, the second LA electrode configured to contact the digit of the left hand and respond to the electrical voltage conducted through the left hand of the user to the second LA electrode.

8. The steering apparatus of claim 1 wherein the curved surface on the left portion and the other curved surface on the right portion are symmetrical with each other across the central portion.

9. The steering apparatus of claim 1 further comprising a temperature sensor positioned with the depressed region of the left portion.

10. The steering apparatus of claim 1 wherein a length of the curved surface is between 20 mm and 60 mm.

11. The steering apparatus of claim 1 wherein a width of the curved surface is between 15 mm and 35 mm.

12. The steering apparatus of claim 1 wherein the curved surface is bounded by a stadium shaped perimeter, wherein a width of the curved surface is between 60% and 65% of a length of the curved surface.

13. The steering apparatus of claim 1 wherein the depressed region is positioned on a top portion of the steering apparatus.

14. The steering apparatus of claim 1 wherein the depressed region is positioned on a front of the steering apparatus facing toward the user.

15. The steering apparatus of claim 1 wherein the depressed region is positioned on a back of the steering apparatus facing away from the user.
